(19) United States
(12) Patent Application Publication
(10) Pub. No.: US 2020/0176131 A1
(43) Pub. Date: Jun. 4, 2020

(54) Systems and Methods for Providing Internet-Based Expert Services

(71) Applicant: Siuvo Inc., Princeton, NJ (US)

(72) Inventor: Bin Shen, Princeton, NJ (US)

(73) Assignee: Siuvo Inc., Princeton, NJ (US)

(21) Appl. No.: 16/615,448

(22) PCT Filed: May 21, 2018
(86) PCT No.: PCT/US18/33705

§ 371 Date: Nov. 21, 2019

Related U.S. Application Data
(60) Provisional application No. 62/509,178, filed on May 21, 2017.

(51) Int. Cl. G16H 80/00; G16H 50/20; G16H 10/20

(52) U.S. Cl. CPC G16H 80/00; G16H 10/20; G16H 50/20

(57) ABSTRACT

This disclosure presents systems and methods for providing Internet-based expert services to users. The systems and methods employs dedicated communication channels, for example, instant message channels, to facilitate service delivery and collaboration between the expert service system and experts.

### CROSS-REFERENCE TO RELATED APPLICATION

This application claims priority under 35 U.S.C. § 119(e) to U.S. Provisional Patent Application Ser. No. 62/509,178, filed on May 21, 2017, the disclosure of which is incorporated herein by reference in its entirety.

### FIELD OF THE INVENTION

The present invention relates generally to systems and methods for providing expert services and more particularly to systems and methods for providing Internet-based expert services to users by employing instant message as communication channels.

### BACKGROUND OF THE INVENTION

Currently, online expert services are provided to users either directly by human experts or by computer software designed by human experts. In the former approach, human experts deliver expert services through web services, which consume human experts significant amount of time on repetitive works. In the latter approach, expert service software designed and/or trained by human experts delivers expert services to users in place of human experts. However, without contributions from human experts, expert service software cannot solve all the problems for users.

Accordingly, there remains a need for systems and methods for providing expert services powered by efficient collaboration between human experts and expert service software through dedicated communication channels.

### SUMMARY OF THE INVENTION

This disclosure addresses this need by providing systems and methods for providing Internet-based expert services. The system includes a non-transitory, computer-readable memory, one or more processors, and a computer-readable medium containing programming instructions. The programming instructions, when executed by the one or more processors, cause the system to receive, from a first user device, a service request from a user through a first communication channel. The service request may include one or more chief reasons related to a condition of a subject and that caused the user to send the service request. The system generates a questionnaire based on the one or more chief reasons for the service request to obtain from the user additional information related to the one or more chief reasons for the service request. The system also transmits the questionnaire to the first user device and prompts the user for a user input to the questionnaire and receives the user input responsive to the questionnaire. The system determines the condition of the subject based on the received user input responsive to the questionnaire and determines a preliminary solution based on the condition of the subject. The system transmits, through a second communication channel to a second user device, the preliminary solution along with the service request and the user input responsive to the questionnaire to a first expert user and prompts the first expert user to provide a first expert response that is a confirmation or a modification of the preliminary solution to the condition of the subject. The second communication channel is different and independent from the first communication channel. The system receives, through the second communication channel from the second user device, the first expert user response to the preliminary solution. The system also generates a final solution to the condition of the subject based on the received first expert user response and presents the final solution to the user by transmitting the final solution to the first user device.

In some embodiments, the subject is the user, the condition relates to a health condition of the user, the first expert user is a healthcare provider, and the final solution to the condition of the subject may include a treatment plan for the subject. In some embodiments, the first communication channel and the second communication channels are instant message channels.

In some embodiments, the system receives a first expert request for involvement of a second expert user and transmits the first expert request and information including a portion of the preliminary solution, the service request, and the user input responsive to the questionnaire, through a third communication channel, to a third user device and prompts a second expert user for a second expert user response that is an opinion of the second expert user based on the received first expert request and the received information. The system receives, from the third user device, the second expert user response and transmits the second expert user response to the second user device. The system also receives, from the second user device, a third expert response from the first expert user based on the received second expert user response. The system further generates the final solution to the condition of the subject based on the received third expert user response and presents the final solution to the user by transmitting the final solution to the first user device.

In some embodiments, the system transmits, to the first user device, a request for a user update from the user as to the current status of the condition of the subject, receives the user update, and determines a progression of the condition of the subject based on the user update. The system also generates a follow-up solution based on the progression of the condition of the subject and transmits the follow-up solution to the first user device.

In some embodiments, the system determines a preliminary follow-up solution based on the progression of the condition of the subject. The system transmits, to the second user device, the preliminary follow-up solution and prompts the expert user for a fourth expert user response based on the user update, the progression of the condition of the subject, and the received preliminary follow-up solution. The system also receives the fourth expert user response from the first expert user, generates the follow-up solution based on the fourth expert user response, and presents the follow-up solution to the user by transmitting the follow-up solution to the first user device.

In some embodiments, the system applies encryption to a communication transmitted through the first communication channel and the second communication channel. The communication may include the service request, the user input, the preliminary solution, the final solution, the first expert response, the second expert response, the third expert response, or the fourth expert response.

This disclosure also provides a method for providing expert services. The method includes receiving, from a first user device, a service request from a user through a first communication channel. The service request may include one or more chief reasons related to a condition of a subject and that caused the user to send the service request. The method also includes generating a questionnaire based on the one or more chief reasons for the service request to obtain from the user additional information related to the one or more chief reasons for the service request. The method includes transmitting the questionnaire to the first user device and prompting the user for a user input to the questionnaire and receiving the user input responsive to the questionnaire. The method also includes determining the condition of the subject based on the received user input responsive to the questionnaire and determining a preliminary solution based on the condition of the subject. The method further includes transmitting, through a second communication channel to a second user device, the preliminary solution along with the service request and the user input responsive to the questionnaire to a first expert user and prompting the first expert user to provide a first expert response that is a confirmation or a modification of the preliminary solution for the condition of the subject. The second communication channel is different and independent from the first communication channel. The method additionally includes receiving, through the second communication channel from the second user device, the first expert user response to the preliminary solution. The method further includes generating a final solution to the condition of the subject based on the received first expert user response and presenting the final solution to the user by transmitting the final solution to the first user device.

In some embodiments, the first expert response from the first expert user may further include a first expert request for involvement of a second expert user and the method includes receiving a first expert request for involvement of a second expert user. The method includes transmitting the first expert request and information comprising a portion of the preliminary solution, the service request, and the user input responsive to the questionnaire, through a third communication channel, to a third user device and prompting a second expert user for a second expert user response that is an opinion of the second expert user based on the received first expert request and the received information. The method also includes receiving, from the third user device, the second expert user response and transmitting the second expert user response to the second user device and receiving, from the second user device, a third expert response from the first expert user based on the received second expert user response. The method further includes generating the final solution to the condition of the subject based on the received third expert user response and presenting the final solution to the user by transmitting the final solution to the first user device.

In some embodiments, the method includes transmitting, to the first user device, a request for a user update from the user as to the current status of the condition of the subject and receiving the user update and determining a progression of the condition of the subject based on the user update. The method also includes generating a follow-up solution based on the progression of the condition of the subject and transmitting the follow-up solution to the first user device.

In some embodiments, the method includes determining a preliminary follow-up solution based on the progression of the condition of the subject. The method includes transmitting, to the second user device, the preliminary follow-up solution and prompting the expert user for a fourth expert user response based on the user update, the progression of the condition of the subject, and the received preliminary follow-up solution. The method includes receiving the fourth expert user response from the first expert user and generating the follow-up solution based on the fourth expert user response and presenting the follow-up solution to the user by transmitting the follow-up solution to the first user device. In some embodiments, the method further includes applying encryption to a communication transmitted through the first communication channel and the second communication channel. The communication may include the service request, the user input, the preliminary solution, the final solution, the first expert response, the second expert response, the third expert response, or the fourth expert response.

According to another aspect of this disclosure, the system for providing expert services receives, from a user device, a first user input from a user that is a service request comprising one or more chief reasons related to a condition of a subject. The system transmits, to a server device through a first communication channel, the first user input. The system receives, from the server device, a questionnaire based on the one or more chief reasons for the service request to obtain from the user additional information related to the one or more chief reasons for the service request. The system also prompts the user to provide a second user input responsive to the questionnaire, transmits the second user input to the server device, and receives, from the service device, a final solution to the condition of the subject, and presents the final solution on a display of the user device. In some embodiments, the final solution is generated based on an expert user input from an expert user received through a second communication channel, the first and second user inputs, and a preliminary determination of the condition of the subject based on the first and second user inputs. In some embodiments, the first communication channel is different and independent from the second communication channel.

According to yet another aspect of this disclosure, the method for providing expert services includes receiving, from a user device, a first user input from a user that is a service request including one or more chief reasons related to a condition of a subject. The method includes transmitting, to a server device through a first communication channel, the first user input. The method includes receiving, from the server device, a questionnaire based on the one or more chief reasons for the service request to obtain from the user additional information related to the one or more chief reasons for the service request. The method also includes prompting the user to provide a second user input responsive to the questionnaire and transmitting the second user input to the server device. The method further includes receiving, from the service device, a final solution to the condition of the subject and presenting the final solution on a display of the user device.

As would be understood by those skilled in the art, all reasonable logical combinations of any embodiments illustrated herein are also encompassed by the present invention.

### BRIEF DESCRIPTION OF THE DRAWINGS

These and other features and advantages of the present invention will become more apparent from the following description when considered in connection with the accompanying drawings, wherein:

FIG. 1 illustrates a schematic diagram generally illustrating an example of an expert service system for providing expert services to users;

FIG. 2 illustrates a sequence chart depicting an example application of an expert service system in which the expert service system seeks inputs from a human expert for providing expert services to users;

FIG. 3 illustrates a sequence chart depicting an example application of an expert service system in which a human expert seeks inputs from the expert service system to provide expert services to users;

FIG. 4 illustrates a sequence chart depicting an example application of an expert service system in which the expert service system coordinates with a human expert to provide expert services to users;

FIG. 5 illustrates a sequence chart depicting an example application of an expert service system in which the expert service system collects information for a human expert to provide expert services to users;

FIG. 6 illustrates a sequence chart depicting an example application of an expert service system in which the expert service system provides psychiatry services to a patient;

FIG. 7 illustrates an exemplary architecture of an expert service system;

FIG. 8 illustrates an exemplary process for providing expert services to users;

FIG. 9 illustrates an exemplary process implemented in a client device to provide expert services to users.

FIG. 10 illustrates an example of application of the expert service system in the psychiatry services to a patient, with the real-time machine generated screen shots shown in FIGS. 10A, 10B, 10C, 10D, 10E, 10F, 10G, 1011, 10I, 10J, 10K, 10L, 10M, and 10N (collectively “FIG. 10”).

### DETAILED DESCRIPTION OF THE INVENTION

This disclosure is not limited to the particular systems, methodologies or protocols described, as these may vary. The terminology used in this description is to describe the particular versions or embodiments only and is not intended to limit the scope.

This disclosure provides systems and methods for providing expert services to users by providing a platform that facilitates service delivery and collaboration between expert service systems and human experts by employing instant message (IM) as communication channels. Through an IM-based communication, the expert service system is able to inquire, provide data to, and receive new tasks from human experts. Moreover, through the IM-based communication, human experts are able to answer, request data from, and deliver new tasks to the expert service system.

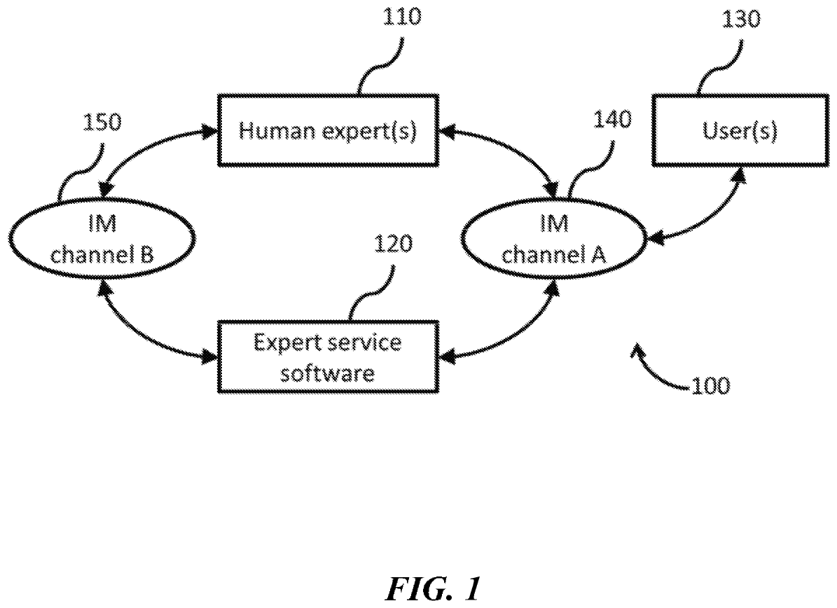

Referring to FIG. 1, the expert service system 100 includes an expert service software 120 and one or more communication channels (e.g., instant message (IM) channel A 140 and instant message (IM) channel B 150). The communication channels assist communication between expert service software 120 and users 130 and between expert service software 120 and service providers (e.g., human experts) 110. The communication channels may be an instant message channels. As understood by a person of ordinary skill in the art, any suitable instant message channels can be utilized to provide a means for real-time communication between expert service software 120 and users 130 and between expert service software 120 and service providers 110. Examples of the instant message channels include, without limitation, WhatsApp, Snapchat, and Facebook massager. Service providers 110 may be experts who have expert knowledge in certain technologies. For example, service providers may include financial specialists, coaches, personal trainers, teachers, musicians, cooks, scientists, engineers, etc. In some embodiments, service providers may also include a healthcare provider (e.g., doctor, nurse, nurse practitioner, clinician, therapist). Unlike conventional online expert services that are provided to users either directly by human experts or by computer software designed by human experts, the expert service system described in this disclosure facilitates and coordinates the collaboration between expert service software 120 and service providers 110 and between service providers. By using instant message channels, the expert service system can push different messages to different users or human experts in a real-time fashion. In contrast, in a conventional client-server architecture, the users or human experts (user ends) need to request updates from the expert software (server end) to retrieve any messages for them. To achieve the “push message” function in a real-time fashion, the user ends need to constantly request updates from the server end. As a result, such expert services provided through a conventional client-server architecture is not scalable. Moreover, if all communications among users, human experts, and expert service software are carried out in one shared IM channel instead of dedicated IM channels, all conversations/messages/information for both users and human experts flowing in the same conversation channel. Consequently, it is difficult for both users and human experts to sort out the useful information. It is inefficient and confusing to handle information exchange in this way. Therefore, dedicated IM channels for different clients (e.g., users, human experts) provide better content segregation, better efficiency, and less confusion.

In addition, the expert service system 100 provides a dynamic way to “push/collect” content to/from users through dedicated IM channels that can be used for two-way or multi-way communication among all participating parties, including users, expert service software, and service providers. In some embodiments, in the case of using instant message channels, the expert service system 100 may employ isolated instant message channels for content segregation. Accordingly, the expert system 100 provides a scalable, flexible, and efficient way for human-computer collaboration.

As used herein, user(s) 130 may include a single user or a group of users of the expert services. Likewise, human expert(s) 110 may include a single human expert or a group of human experts to provide expert services. In some embodiments, the expert service software 120 may include an artificial intelligence robot (“AI robot”). Methods to implement the AI robot may include: (1) rule-based configuration, (2) Petri-net based graph implementation, (3) business processing model and notation, and (4) neural network based algorithm with adaptive machine learning and notation.

As illustrated in FIG. 1, the expert service system delivers expert services to user(s) 130 through one or more communication channels. The expert services may be initiated by user(s) 130 by submitting a service request to expert service software 120. Alternatively, the expert services may be initiated by service providers, i.e., human expert(s) 110, or by expert service software 120. In one example, the communication channels may include IM channel A 140. The expert services may be contributed by one or more human expert(s) 110 or expert service software 120. In some embodiments, the communications between human expert(s) 110 and user(s) 130, between expert service software 120 and user(s) 130, and among user(s) 130 are all through IM channel A 140. In the event that human expert(s) 110 or expert service software 120 need to exchange information, seek help or establish other forms of collaborations with expert service software 120 or other human expert(s) 110, the communication for the collaborations is realized through IM channel B 150. The communications among human expert(s) 110 also take place through IM channel B 150. In some embodiments, IM channel A is different and independent from IM channel B, such that user(s) 130 cannot access IM channel B 150 that is dedicated for the use by human expert(s) 110 and expert service software 120.

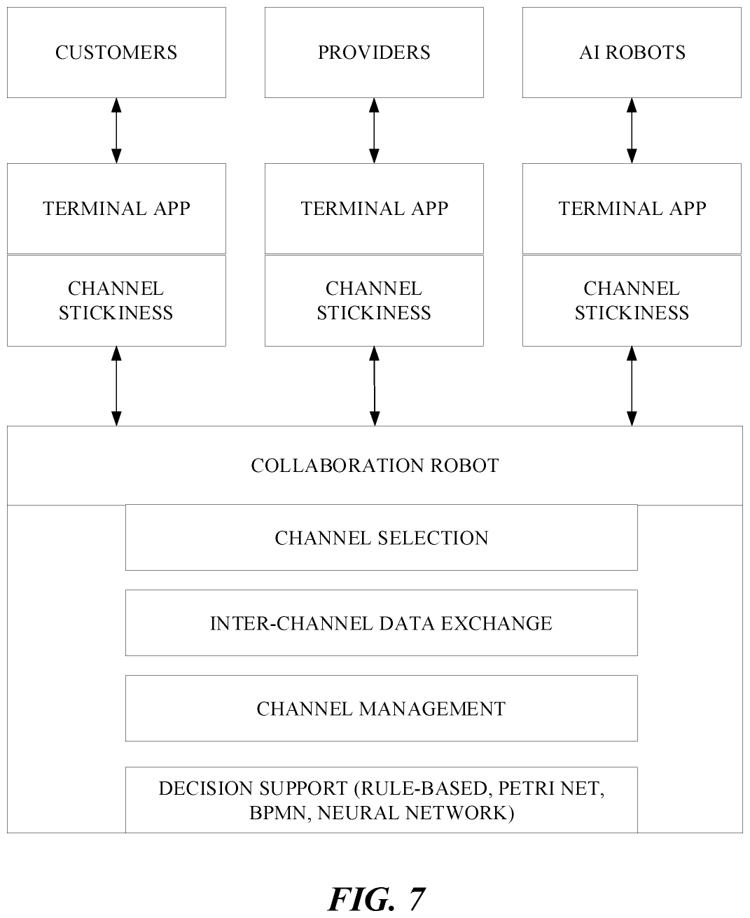

In some embodiments, as illustrated in FIG. 7, the expert service system provides expert services by a multi-directional information exchange or bi-directional information exchange, such that (1) users also receive information from the service providers and the robot, and (2) there is information exchange between the robot and the service provider. In one example, for the information exchange taking place as a group-wise communication, the information is visible to all participating parties, including the user (e.g., client, customer), the service provider and the AI robot or to sub-groups of the participating parties. In some embodiments, the information exchange takes place as a peer-wise communication, where the information is exchanged only between two parties in the group. The expert service system provides the group-wise communication and peer-wise communication in separate IM channels to ensure content segregation and scalability.

The expert service system 100 also provides an efficient implementation to: (1) switch communication channel during a service session; (2) provide information barrier between the communication channels; and (3) exchange information between the channels. In some embodiments, the expert service system carries out the implementation of (1) and (3) by engaging the AI robot associated with the expert service software.

By way of non-limiting examples, the expert service system is further described. In some embodiments, the expert service system may provide expert services in the healthcare industry. For example, a patient may start a service session with the physician through the expert service system. First, the expert service system sends a greeting message to the patient by engaging a group-wise communication. Second, the expert service system sends instructions to the patient by engaging a peer-wise communication between the expert service software (i.e., the AI robot) and the patient. The expert service system also sends a request to ask the patient to fill out chief complaints (i.e., the reason for this hospital visit) via a group-wise communication. The patient responds to the request by submitting the chief complaints, for example, dysphagia, via a group-wise communication. Based on the submitted the chief complaints, the expert service system inquires additional symptoms from the patient related to dysphagia also via group-wise communication. The expert service system sends potential diagnosis and treatment plan to the physician via clinic decision support (CDS) via a peer-wise communication between the AI robot and the physician. The physician reviews and revises the diagnosis and treatment via a peer-wise communicate between the AI robot and the physician. With the assistant of the AI robot, the physician may invite a radiologist for an additional opinion on a test result (e.g., CT scan) via a sub-group communication between the robot, the physician, and the specialist. In this embodiment, the specialists are temporally invited to join the session, have access to necessary information, and then leave the session. The physician then communicates the final diagnosis to the patient and recommended treatment plan via a group-wise communication.

In some embodiments, the expert service system may track the efficacy of treatment via a peer-wise communication between the robot and the patient. As understood by a person of ordinary skill in the art, there can be many permutations of how group-wise/peer-wise/subgroup-wise communication during a specific healthcare service based on the problem in hand, the hospital workflow, the clinic path of a disease, the patient desire, and the physician's preference, etc.

In some embodiments, for cybersecurity purposes, the expert service system may apply encryption to the exchanged information transmitted through the communication channels. In some embodiments, data are encrypted at terminal apps, including user apps (i.e., patients app, physician app) or server apps on the application server hosting the AI robot to ensure secure communication via the Internet to protect privacy.

Data containing exchanged information among participating parties including users, service providers, and expert service software, may be structured data or unstructured data. In some embodiments, the expert service system may implement an additional layer at the terminal app to transform human-friendly data (usually unstructured data, i.e., natural language) to machine friendly data (structured data). Alternatively and/or optionally, the expert service system may transform the machine-friendly data to human-friendly data such that users or service providers would have a better understanding in the exchanged information.

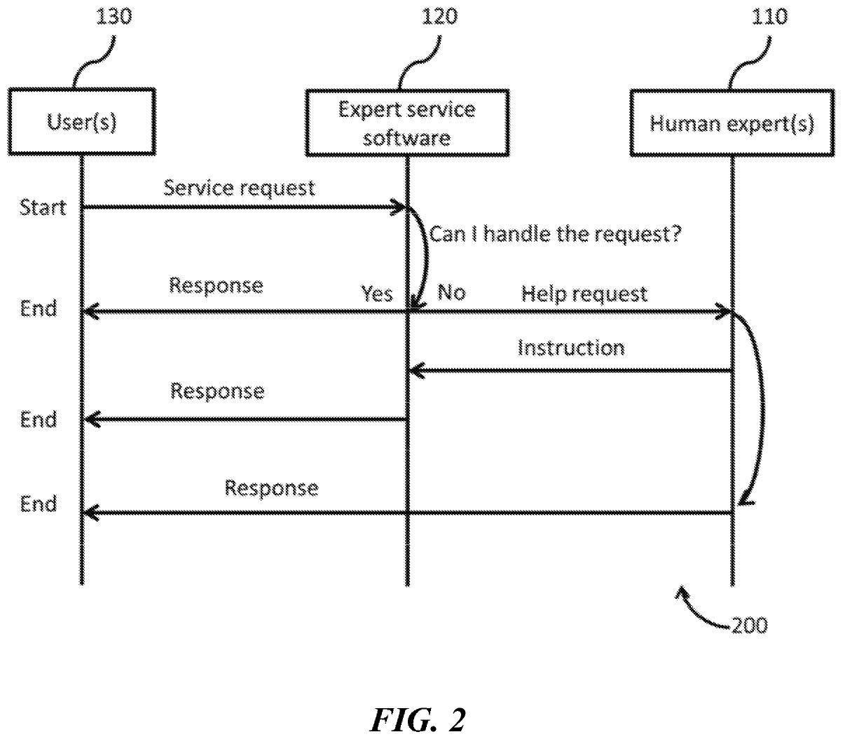

Referring now to FIG. 2, the sequence chart illustrates an example application 200 of expert service system 100, in which expert service software 120 needs inputs from human expert(s) 110. To start, user(s) 130 sends a service request to expert service software 120. Upon receiving the request, expert service software 120 evaluates whether or not it can directly provide solutions to the request. In the event that the expert service software determines that it is capable of providing solutions to the service request, expert service software 120 sends a response to user(s) 130 to address the service request, which ends the expert service. On the other hand, if the expert service software determines that it is incapable of providing solutions to the service request, then expert service software 120 sends a request to human expert(s) 110. Upon receiving the request from expert service software, the human expert(s) 110 may choose to engage one of two options: (1) the human expert(s) 110 may send an instruction to expert service software 120. With the instruction, expert service software 120 is able to handle the service request from user(s) 130, and sends its response to user(s) 130, which ends the expert service; (2) the human expert(s) 110 send response directly to user(s) 130, which ends the expert service.

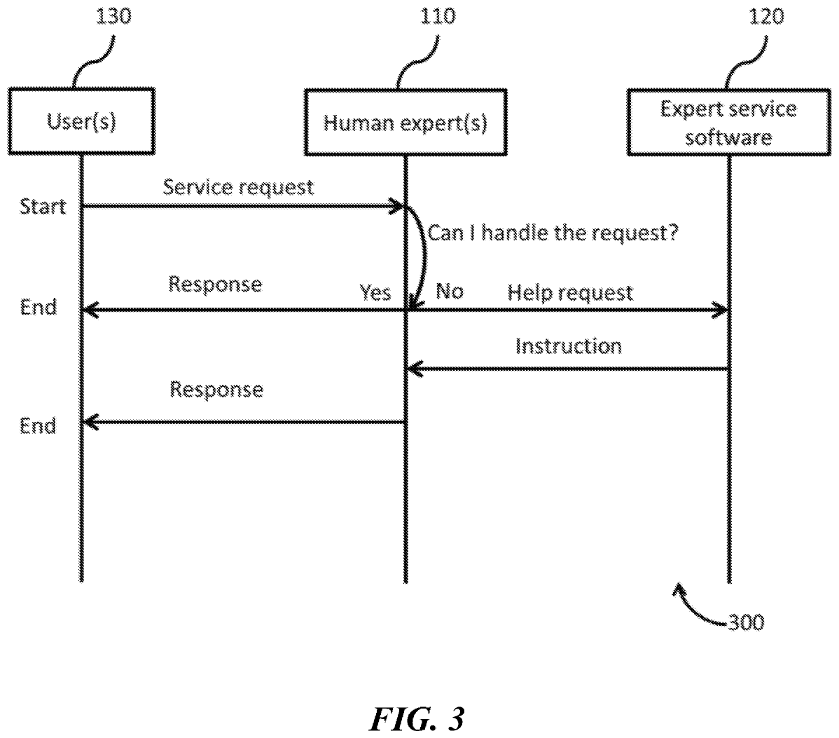

Referring to FIG. 3, the sequence chart illustrates an example application 300 of expert service system 100, in which human expert(s) 110 needs inputs from expert service software 120. To start, user(s) 130 sends a service request to human expert(s) 110. Upon receiving the request, human expert(s) 110 evaluates whether or not he/she can provide solutions to the request. If human expert(s) 110 determines that he/she is capable of providing solutions to the request, then human expert(s) 110 sends a response to user(s) 130 to address the service request, which ends the expert service. On the other hand, if human expert(s) 110 determines that he/she is incapable of providing solutions to the request, then human expert(s) 110 sends a request to expert service software 120. After receiving the request, expert service software 120 sends an instruction to human expert(s) 110. With the instruction, human expert(s) 110 sends a response to user(s) 130, which ends the expert service.

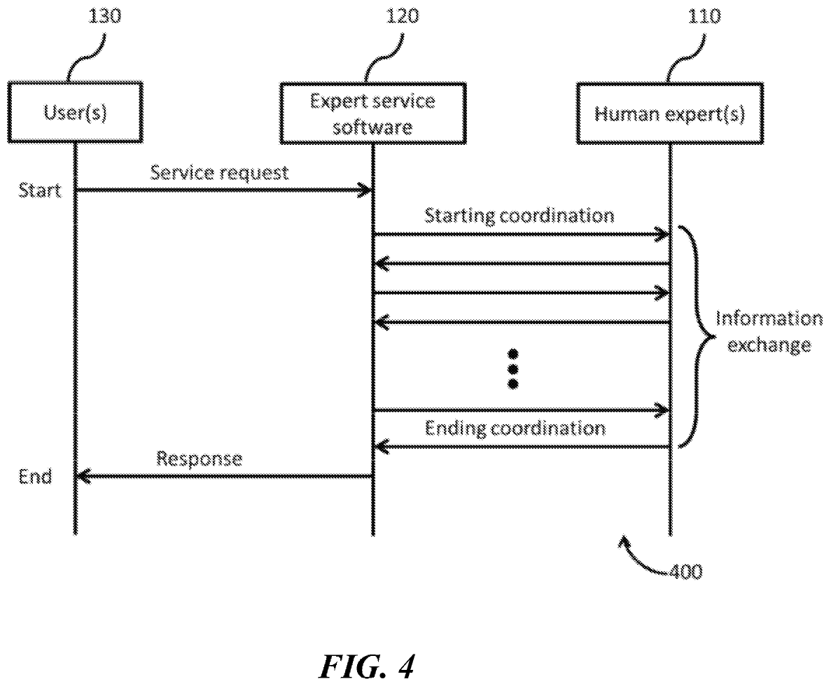

Referring to FIG. 4, the sequence chart illustrates an example application 400 of expert service system 100, in which expert service software 120 needs coordination with human expert(s) 110 to deliver expert services. To start, user(s) 130 sends a service request to expert service software 120. After receiving the request, expert service software 120 sends a request to human expert(s) 110 to start a collaboration. During the collaboration, the expert service system engages rounds of communication between human expert(s) 110 and expert service software 120, which includes data sharing, questions/answers, and task requests/responses. The human expert(s) 110 may send a message to expert service software 120 to end the collaboration. Then expert service software 120 sends respond to user(s) 130, which ends the expert service.

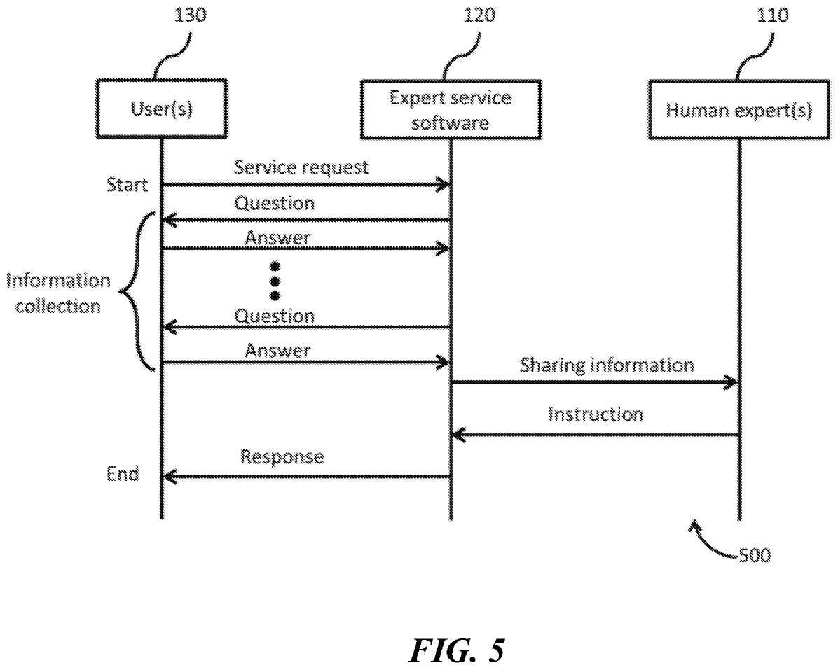

Referring to FIG. 5, the sequence chart illustrates an example application 500 of expert service system 100, in which expert service software 120 collects information for human expert(s) 110 to deliver the expert service. To start, user(s) 130 sends a service request to expert service software 120. After receiving the request, expert service software 120 starts to collect information by asking a battery of questions to user(s) 130 and receiving answers from user(s) 130. After all the information needed is collected, expert service software 120 sends the information to human expert(s) 110. Then human expert(s) 110 sends an instruction to expert service software 120. According to the instruction, expert service software 120 sends a response to user(s) 130, which ends the expert service.

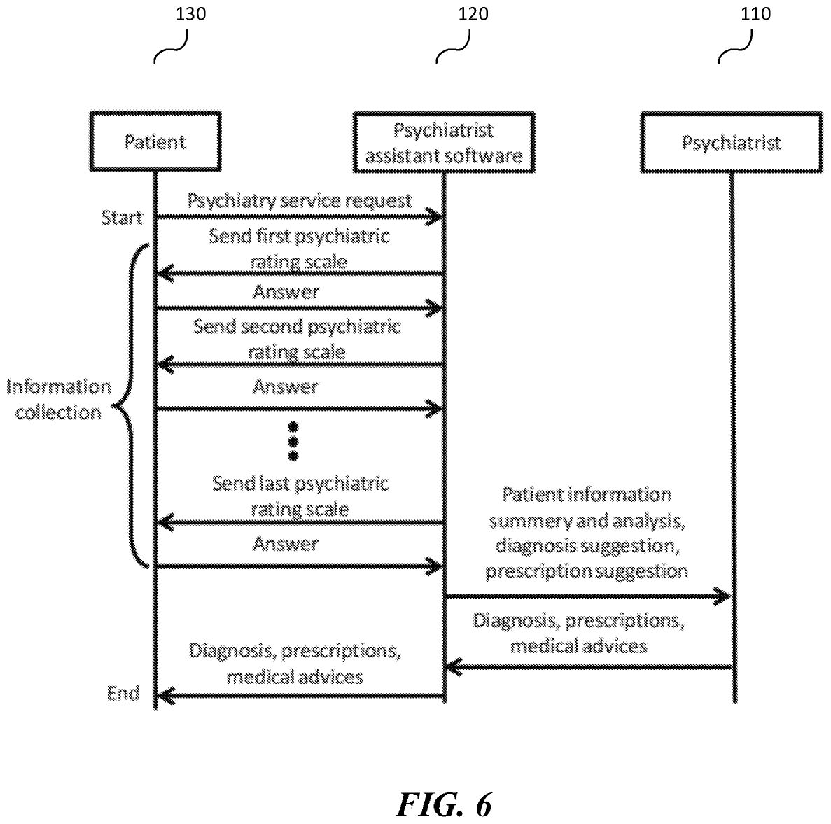

Referring to FIG. 6, the sequence chart illustrates an example application 600 of expert service system 100 in providing psychiatry services. The expert service system includes psychiatrist assistant software 120 that collects information from patients for psychiatrist 110 in order to deliver the psychiatrist service. To start, the patient 130 sends a psychiatry service request to psychiatrist assistant software 120. Upon receiving the psychiatrist service request, psychiatrist assistant software 120 starts to collect information by providing the first psychiatric rating scale to patient 130 and receiving from patient 130 answers responsive to the first psychiatric rating scale. Based on the answers from the patient 130 to the first psychiatric rating scale, psychiatrist assistant software 120 may send a second psychiatric rating scale to patient 130 to collect additional information. Such iterations for information exchange between psychiatrist 120 and patient 130 may continue until psychiatrist assistant software 120 determines that sufficient information has been collected to warrant a diagnosis for a psychiatric condition of patient 130. Based on the information collected from patient 130, psychiatrist assistant software 120 may generate a diagnosis and/or prescription suggestion for the psychiatric condition of patient 130 and send the diagnosis and/or prescription suggestion along with patient information, summary, and analysis of the collected information to psychiatrist 110. Psychiatrist 110 sends an instruction containing diagnosis, prescriptions, and medical advice to psychiatrist assistant software 120. According to the instruction, psychiatrist assistant software 120 sends a response to patient 130, which ends the expert service.

For the examples illustrated in FIGS. 2-6, all the communication between user(s) 130 and expert service software 120 takes place through IM channel A 140. All the communication between human expert(s) 110 and expert service software 120 takes place through IM channel B 150. All the communication between human expert(s) 110 and user(s) 130 takes place through IM channel A 140.

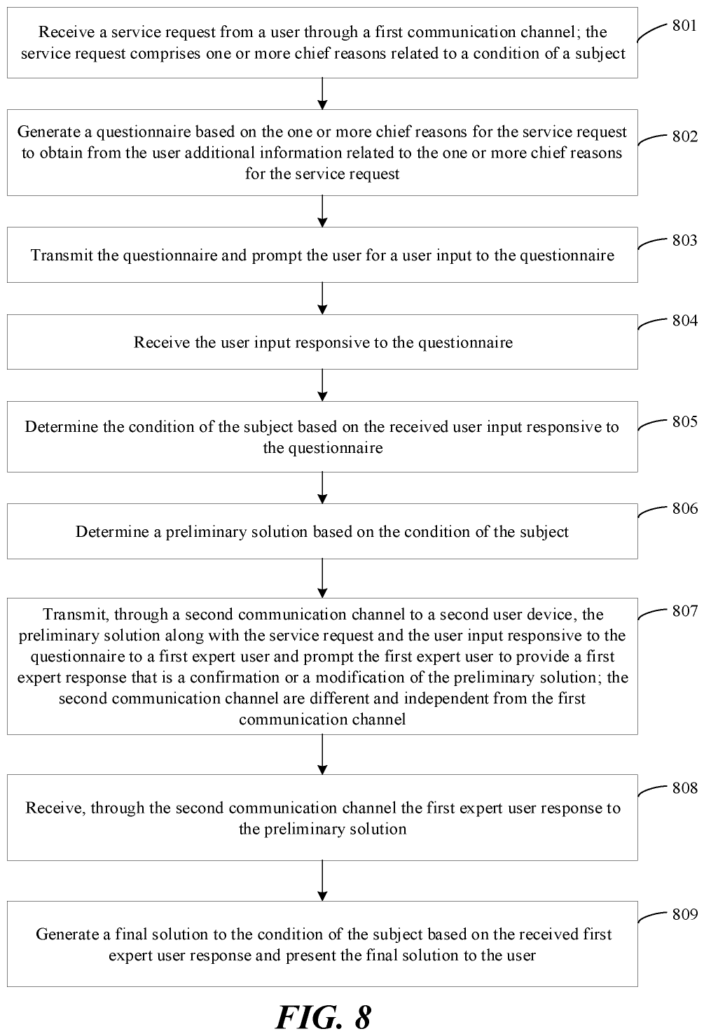

FIG. 8 illustrates an example of a system for providing expert services. At 801, the system receives, from a first user device, a service request from a user through a first communication channel. The service request includes one or more chief reasons related to a condition of a subject and that caused the user to send the service request. At 802, the system generates a questionnaire based on the one or more chief reasons for the service request to obtain, from the user, additional information related to the one or more chief reasons for the service request. At 803, the system also transmits the questionnaire to the first user device and prompts the user for a user input to the questionnaire and receives the user input responsive to the questionnaire at 804. At 805, the system determines the condition of the subject based on the received user input responsive to the questionnaire, and at 806 the system determines a preliminary solution based on the condition of the subject. At 807, the system transmits, through a second communication channel to a second user device, the preliminary solution along with the service request and the user input responsive to the questionnaire to a first expert user and prompts the first expert user to provide a first expert response that is a confirmation or a modification of the preliminary solution for the condition of the subject. The second communication channel is different and independent from the first communication channel. At 808, the system receives, through the second communication channel from the second user device, the first expert user response to the preliminary solution. At 809, the system also generates a final solution to the condition of the subject based on the received first expert user response and presents the final solution to the user by transmitting the final solution to the first user device.

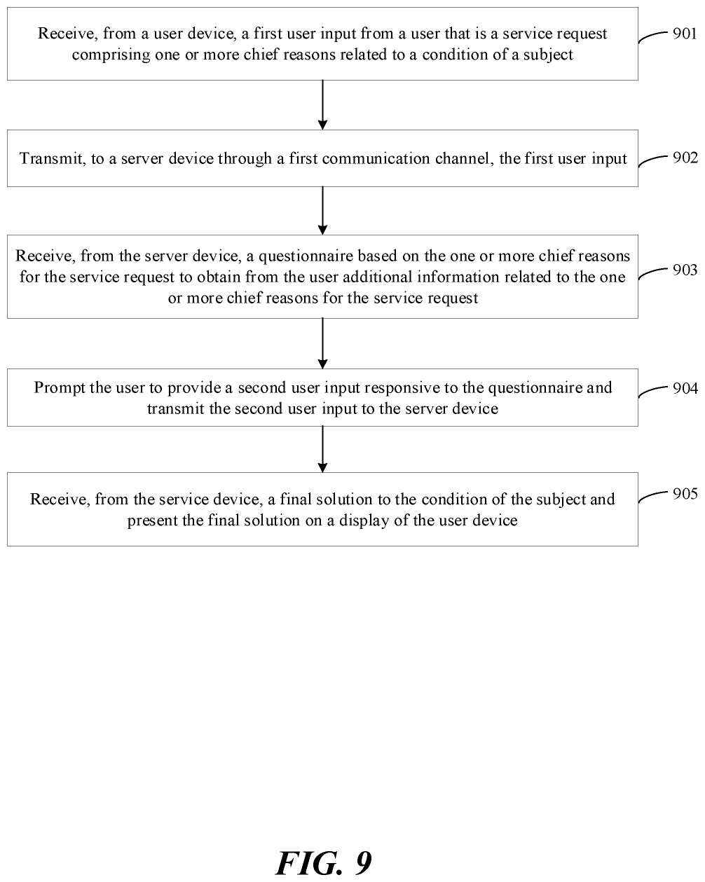

FIG. 9 illustrates an example of a system implemented on a client device for providing expert services. At 901, the system receives, from a user device, a first user input from a user that is a service request comprising one or more chief reasons related to a condition of a subject. At 902, the system transmits, to a server device through a first communication channel, the first user input. At 903, the system receives, from the server device, a questionnaire based on the one or more chief reasons for the service request to obtain from the user additional information related to the one or more chief reasons for the service request. At 904, the system prompts the user to provide a second user input responsive to the questionnaire and transmits the second user input to the server device. At 905, the system receives, from the service device, a final solution to the condition of the subject and presents the final solution on a display of the user device.

### Example

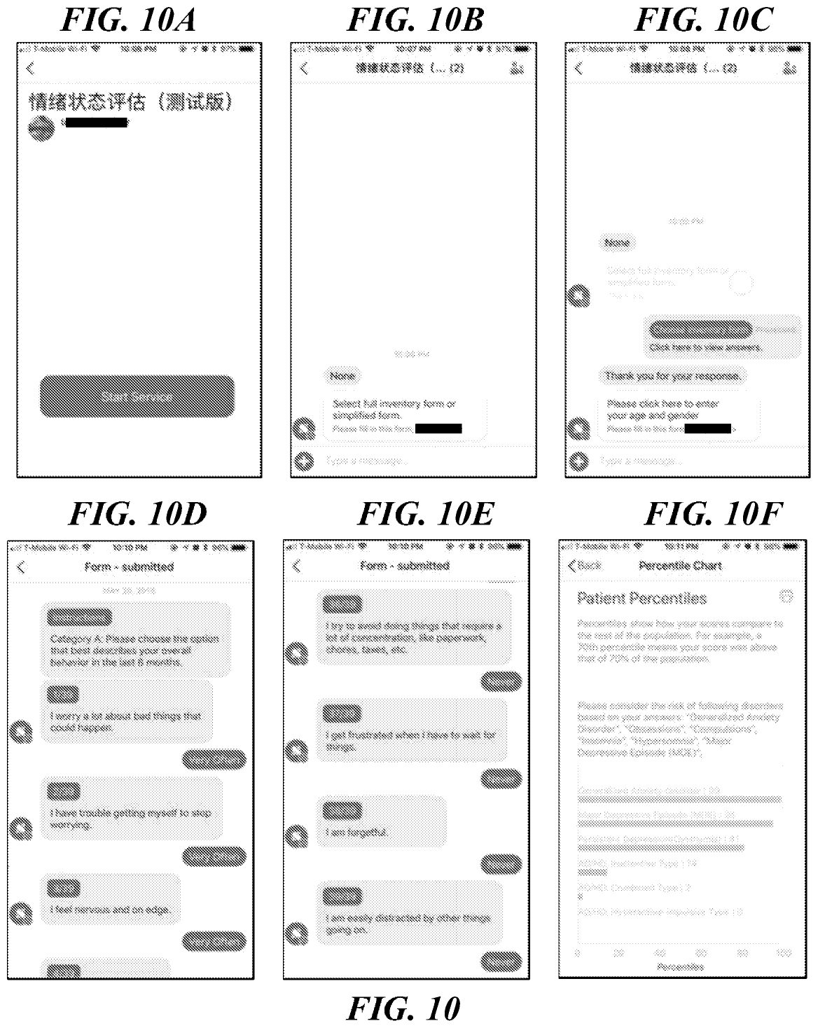

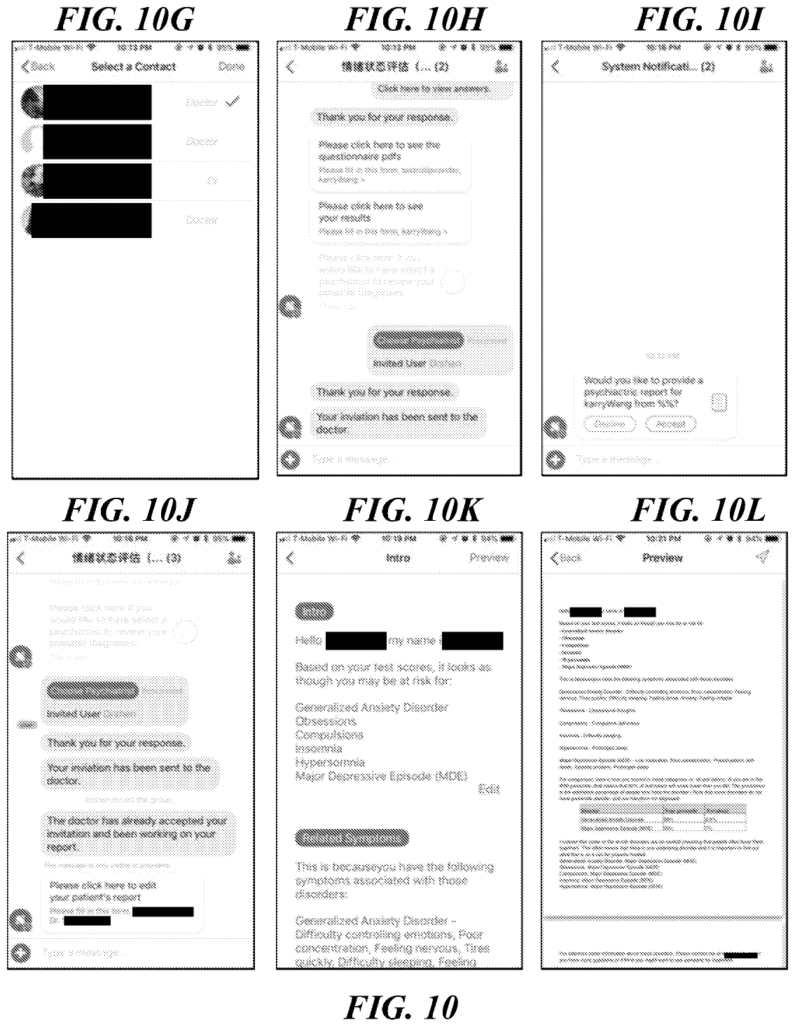

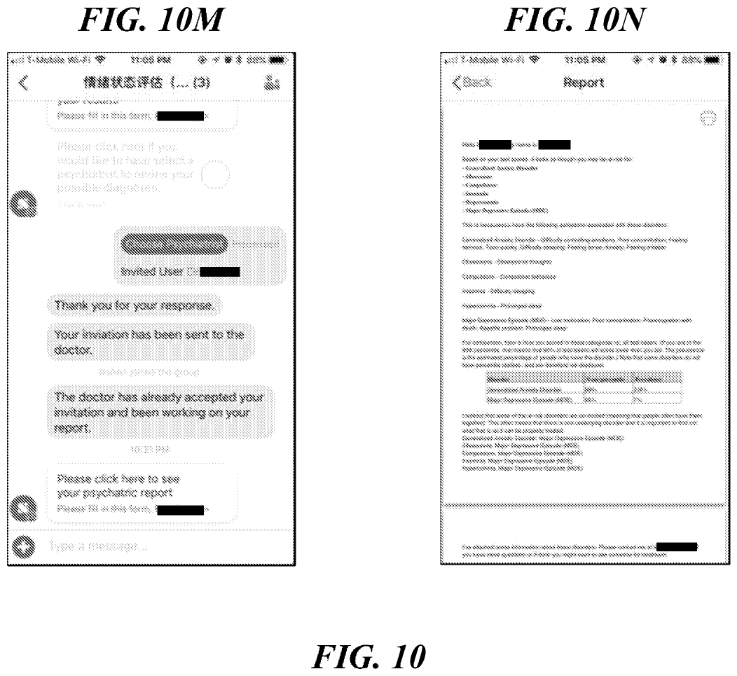

FIG. 10 includes screenshots demonstrating an example application of an expert service system in which the expert service system provided psychiatry services to a patient. As shown in FIG. 10A, the service expert system received a service request for mental health evaluation from the patient. The expert service system prompted the patient to choose whether he/she would like to take a full or simplified evaluation for mental health (FIG. 10B). The patient chose the simplified form to proceed with the evaluation. The expert service system sent the patient a list of general questions (e.g., age, gender), as shown in FIG. 10C, and a psychiatric rating scale (e.g., simplified version that includes instructions and 39 questions). The patient completed the scale by answering the questions from the expert service system one by one (FIGS. 10D and 10E). Upon receiving the answers responsive to the general questions and the psychiatric rating scale, the expert service system sent the patient the psychiatric rating scale summery and evaluation (FIG. 10F). The evaluation results were provided in the form of percentiles in which the patient's scores were compared to the rest of the population. For example, a 70^{th} percentile means the patient's score was above that of 70% of the population. As shown in FIG. 10F, based on the patient's scores, the expert service system alerted the patent the risk of one or more disorders, for example, generalized anxiety disorder, obsessions, compulsions, insomnia, hypersomnia, major depressive episode (MIRE).

Because the evaluation results suggested potential risks of mental diseases, the expert service system sent the patient a list of psychiatrists to provide a psychiatric report for further evaluation (FIG. 10G). After the patient selected a psychiatrist from the list of psychiatrists, the expert service system informed the patient that an invitation had been sent to the selected psychiatrist (FIG. 1011) and sent an invitation to the psychiatrist (FIG. 10I). After the psychiatrist accepted the invitation, the expert service system sent him/her patient's information (e.g., the psychiatric rating scale summery and the evaluation result). The expert service system also generated a diagnosis report based on the evaluation result and sent it to the psychiatrist for review and revision (FIG. 10J). The psychiatrist edited the diagnosis report (FIG. 10K), reviewed the final diagnosis report in PDF format (FIG. 10L), and submitted the report to the expert service system. After receiving the report from the psychiatrist, the expert service system sent the final diagnosis report to the patient (FIG. 10M). The patient reviewed the report in PDF (FIG. 10N). The expert service system also provided the patient an option to print out the report or to start a message/video chat with the psychiatrist if he/she has further questions.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

Other objects, features, and advantages of the present invention will become apparent from the detailed description and examples described above and the follow claims. It should be understood, however, that the detailed description and the examples, while indicating specific embodiments of the invention, are given by way of illustration only. Additionally, numerous variants and modifications within the spirit and scope of the invention apparent to those skilled in the art are also contemplated by the present invention as reflected in the summary of the invention and the following claims.

1. A system for providing expert services, comprising:
a non-transitory, computer readable memory;
one or more processors; and
a computer-readable medium containing programming instructions that, when executed by the one or more processors, cause the system to:
receive, from a first user device, a service request from a user through a first communication channel, wherein the service request comprises one or more chief reasons related to a condition of a subject and that caused the user to send the service request;
generate a questionnaire based on the one or more chief reasons for the service request to obtain, from the user, additional information related to the one or more chief reasons for the service request;
transmit the questionnaire to the first user device and prompt the user for a user input to the questionnaire;
receive the user input responsive to the questionnaire;
determine the condition of the subject based on the received user input responsive to the questionnaire;
determine a preliminary solution based on the condition of the subject;
transmit, through a second communication channel to a second user device, the preliminary solution along with the service request and the user input responsive to the questionnaire to a first expert user and prompt the first expert user to provide a first expert response that is a confirmation or a modification of the preliminary solution for the condition of the subject, wherein the second communication channel is different and independent from the first communication channel;
receive, through the second communication channel from the second user device, the first expert user response to the preliminary solution; and
generate a final solution to the condition of the subject based on the received first expert user response and present the final solution to the user by transmitting the final solution to the first user device.

2. The system of claim 1, wherein the first and the second communication channels are instant message channels.

3. The system of claim 1, wherein the subject is the user, and the condition relates to a health condition of the user, and the first expert user is a healthcare provider, and wherein the final solution to the condition of the subject comprises a treatment plan for the subject.

4. The system of claim 1, further comprising programming instructions configured to:
receive a first expert request for involvement of a second expert user;
transmit the first expert request and information comprising a portion of the preliminary solution, the service request, and the user input responsive to the questionnaire, through a third communication channel, to a third user device and prompt a second expert user for a second expert user response that is an opinion of the second expert user based on the received first expert request and the received information;
receive, from the third user device, the second expert user response and transmit the second expert user response to the second user device;
receive, from the second user device, a third expert response from the first expert user based on the received second expert user response; and
generate the final solution to the condition of the subject based on the received third expert user response and present the final solution to the user by transmitting the final solution to the first user device.

5. The system of claim 1, further comprising programming instructions configured to:
transmit, to the first user device, a request for a user update from the user as to the current status of the condition of the subject;
receive the user update and determine a progression of the condition of the subject based on the user update;
generate a follow-up solution based on the progression of the condition of the subject; and
transmit the follow-up solution to the first user device.

6. The system of claim 1, further comprising programming instructions configured to:
determine a preliminary follow-up solution based on the progression of the condition of the subject;
transmit, to the second user device, the preliminary follow-up solution and prompt the expert user for a fourth expert user response based on the user update, the progression of the condition of the subject, and the received preliminary follow-up solution;
receive the fourth expert user response from the first expert user and generate the follow-up solution based on the fourth expert user response; and
present the follow-up solution to the user by transmitting the follow-up solution to the first user device.

7. The system of claim 1, further comprising programming instructions configured to:
apply encryption to a communication transmitted through the first communication channel and the second communication channel, wherein the communication comprises the service request, the user input, the preliminary solution, the final solution, the first expert response, the second expert response, the third expert response, or the fourth expert response.

8. A method for providing expert services, comprising:
receiving, from a first user device, a service request from a user through a first communication channel, wherein the service request comprises one or more chief reasons related to a condition of a subject and that caused the user to send the service request;
generating a questionnaire based on the one or more chief reasons for the service request to obtain from the user additional information related to the one or more chief reasons for the service request;
transmitting the questionnaire to the first user device and prompting the user for a user input to the questionnaire;
receiving the user input responsive to the questionnaire;
determining the condition of the subject based on the received user input responsive to the questionnaire;
determining a preliminary solution based on the condition of the subject;
transmitting, through a second communication channel to a second user device, the preliminary solution along with the service request and the user input responsive to the questionnaire to a first expert user and prompting the first expert user to provide a first expert response that is a confirmation or a modification of the preliminary solution for the condition of the subject, wherein the second communication channel is different and independent from the first communication channel;
receiving, through the second communication channel from the second user device, the first expert user response to the preliminary solution; and
generating a final solution to the condition of the subject based on the received first expert user response and presenting the final solution to the user by transmitting the final solution to the first user device.

9. The method of claim 8, wherein the first and the second communication channels are instant message channels.

10. The method of claim 8, wherein the subject is the user and the condition relates to a health condition of the user, and the first expert user is a healthcare provider, and wherein the final solution to the condition of the subject comprises a treatment plan for the subject.

11. The method of claim 8, wherein the first expert response from the first expert user further comprises a first expert request for involvement of a second expert user, and the method further comprises:
receiving a first expert request for involvement of a second expert user;
transmitting the first expert request and information comprising a portion of the preliminary solution, the service request, and the user input responsive to the questionnaire, through a third communication channel, to a third user device and prompting a second expert user for a second expert user response that is an opinion of the second expert user based on the received first expert request and the received information;
receiving, from the third user device, the second expert user response and transmitting the second expert user response to the second user device;
receiving, from the second user device, a third expert response from the first expert user based on the received second expert user response; and
generating the final solution to the condition of the subject based on the received third expert user response and presenting the final solution to the user by transmitting the final solution to the first user device.

12. The method of claim 8, further comprising:
transmitting, to the first user device, a request for a user update from the user as to the current status of the condition of the subject;
receiving the user update and determining a progression of the condition of the subject based on the user update;
generating a follow-up solution based on the progression of the condition of the subject; and
transmitting the follow-up solution to the first user device.

13. The method of claim 12, wherein generating a follow-up solution comprises:
determining a preliminary follow-up solution based on the progression of the condition of the subject;
transmitting, to the second user device, the preliminary follow-up solution and prompting the expert user for a fourth expert user response based on the user update, the progression of the condition of the subject, and the received preliminary follow-up solution;
receiving the fourth expert user response from the first expert user and generating the follow-up solution based on the fourth expert user response; and
presenting the follow-up solution to the user by transmitting the follow-up solution to the first user device.

14. The method of claim 8, further comprising:
applying encryption to a communication transmitted through the first communication channel and the second communication channel, wherein the communication comprises the service request, the user input, the preliminary solution, the final solution, the first expert response, the second expert response, the third expert response, or the fourth expert response.

15. A system for providing expert services, comprising:
a non-transitory, computer readable memory;
one or more processors; and
a computer-readable medium containing programming instructions that, when executed by the one or more processors, cause the system to:
receive, from a user device, a first user input from a user that is a service request comprising one or more chief reasons related to a condition of a subject;
transmit, to a server device through a first communication channel, the first user input;
receive, from the server device, a questionnaire based on the one or more chief reasons for the service request to obtain from the user additional information related to the one or more chief reasons for the service request;
prompt the user to provide a second user input responsive to the questionnaire and transmit the second user input to the server device; and
receive, from the service device, a final solution to the condition of the subject and present the final solution on a display of the user device.

16. The system of claim 15, wherein the final solution is generated based on an expert user input from an expert user received through a second communication channel, the first and second user inputs, and a preliminary determination of the condition of the subject based on the first and second user inputs, wherein the first communication channel is different and independent from the second communication channel.

17. The system of claim 15, wherein the subject is the user, the condition relates to a health condition of the subject, and the expert user is a healthcare provider, and wherein the final solution to the condition of the subject comprises a treatment plan for the subject.

18. A method for providing expert services using the system of claim 15, comprising:
receiving, from a user device, a first user input from a user that is a service request comprising one or more chief reasons related to a condition of a subject;
transmitting, to a server device through a first communication channel, the first user input;
receiving, from the server device, a questionnaire based on the one or more chief reasons for the service request to obtain from the user additional information related to the one or more chief reasons for the service request;
prompting the user to provide a second user input responsive to the questionnaire and transmitting the second user input to the server device; and
receiving, from the service device, a final solution to the condition of the subject and presenting the final solution on a display of the user device.

19. The method of claim 18, wherein the final solution is generated based on an expert user input from an expert user received through a second communication channel, the first and second user inputs, and a preliminary determination of the condition of the subject based on the first and second user inputs, wherein the first communication channel is different and independent from the second communication channel.

20. The method of claim 18, wherein the subject is the user, the condition relates to a health condition of the subject, and the expert user is a healthcare provider, and wherein the final solution to the condition of the subject comprises a treatment plan for the subject.
